(19) United States
(12) Patent Application Publication
(10) Pub. No.: US 2024/0037494 A1
(43) Pub. Date: Feb. 1, 2024

(54) DIETARY FOOD DELIVERY SYSTEM TAILORED TO CONSUMER'S EATING PATTERNS

(71) Applicant: Tae Hoon YOON, Seoul (KR)

(72) Inventor: Tae Hoon YOON, Seoul (KR)

(21) Appl. No.: 17/966,430

(22) Filed: Oct. 14, 2022

(30) Foreign Application Priority Data
Jul. 28, 2022 (KR) 10-2022-0094045

(51) Int. Cl. G06Q 10/08; G06Q 10/04; G16H 20/60

(52) U.S. Cl. CPC G06Q 10/08355; G06Q 10/0832; G06Q 10/0833; G06Q 10/047; G06Q 10/087; G16H 20/60

(57) ABSTRACT

Disclosed herein is a food delivery system tailored to a consumer's eating patterns, which determines priorities of delivery according to a consumer's meal time to set a delivery line when delivering dietary food to a consumer, and transfers dietary food to a deliverer of a neighboring sector in a case in which it is difficult to deliver dietary food in time. The food delivery system includes: a dietary food registration unit for registering dietary food classified into a plurality of types; a food order receiving unit for receiving a delivery order including type of the dietary food and a meal cycle and collecting eating patterns including meal time; a delivery target establishment unit for establishing target time of the corresponding food including the type of the dietary food to be delivered and a start time and an end time of a delivery period by using the delivery order and the eating patterns of each consumer; a deliverer registration unit for dividing a delivery district into a plurality of delivery zones and allocating and registering each deliverer to each delivery zone; a delivery schedule unit for determining priorities of consumers with respect to consumers' delivery destinations assigned to each deliverer and selecting a consumer having the highest priority as a consumer of the next delivery destination; and a food transfer processing unit for determining whether a specific deliverer can deliver the corresponding dietary food to the corresponding consumer within the target time, and transferring the delivery for the corresponding consumer to a neighboring deliverer when the specific deliverer cannot deliver the corresponding dietary food to the corresponding consumer.

The food delivery system can determine priorities of delivery according to a consumer's eating patterns, thereby delivering dietary food to each consumer at the closest time to the consumer's desired meal time, and maintaining the best freshness state of the dietary food.

### BACKGROUND OF THE INVENTION

### Field of the Invention

The present invention relates to a food delivery system tailored to a consumer's eating patterns, which determines priorities of delivery according to a consumer's meal time to set a delivery line when delivering dietary food to a consumer, and transfers dietary food to a deliverer of a neighboring sector in a case in which it is difficult to deliver dietary food in time.

### Background Art

In general, E-commerce which is an abbreviation of electronic commerce to buy and sell goods and services through an online network. With the development of smart phones and logistics/distribution systems, the proportion of shopping using the E-commerce is increasing.

In addition, even groceries which have high proportion in offline sales are increasing in sales in the online market. Moreover, with an increase of one-person households and an increase of interest in cooking, sales of food is continuously increasing.

In particular, in the case of food, in order to maintain freshness, a same-day delivery service, such as early-morning delivery, has been increased. Accordingly, a technology for delivering a fruit or the like in the morning has been proposed (Patent Literature 1).

However, most of the conventional food delivery methods aim to deliver food early in the morning or at specific time of the morning. Therefore, the conventional food delivery methods never consider a consumer's meal time or life patterns.

For instance, a consumer A gets up at dawn and has a meal at six in the morning, but a consumer B gets up late and has a meal at nine in the morning. If food is delivered before 6 a.m., the consumer A can always receive fresh food, but the consumer B receives food three hours later. So, the food that the consumer B received loses freshness. Especially, if a delivery vehicle equipped with a refrigeration facility or the like delivers food to the consumer's door, after the delivery, freshness of the food may be deteriorated. Furthermore, if food is delivered by seven a.m., the consumer A cannot have a meal at the time the consumer wants.

Therefore, technology of delivering food at the time each consumer wants.

### PATENT LITERATURE

### Patent Documents

Patent Document 1: Korean Patent Laid-open No. 10-2004-0050717 (published on Jun. 17, 2004)

### SUMMARY OF THE INVENTION

Accordingly, the present invention has been made to solve the above-mentioned problems occurring in the prior arts, and it is an object of the present invention to provide a food delivery system tailored to a consumer's eating patterns, which determines priorities of delivery according to a consumer's meal time to set a delivery line when delivering dietary food to a consumer, and transfers dietary food to a deliverer of a neighboring sector in a case in which it is difficult to deliver dietary food in time.

To accomplish the above object, according to the present invention, there is provided a food delivery system tailored to a consumer's eating patterns including: a dietary food registration unit for registering dietary food classified into a plurality of types; a food order receiving unit for receiving a delivery order including type of the dietary food and a meal cycle and collecting eating patterns including meal time; a delivery target establishment unit for establishing target time of the corresponding food including the type of the dietary food to be delivered and a start time and an end time of a delivery period by using the delivery order and the eating patterns of each consumer; a deliverer registration unit for dividing a delivery district into a plurality of delivery zones and allocating and registering each deliverer to each delivery zone; a delivery schedule unit for determining priorities of consumers with respect to consumers' delivery destinations assigned to each deliverer and selecting a consumer having the highest priority as a consumer of the next delivery destination; and a food transfer processing unit for determining whether a specific deliverer can deliver the corresponding dietary food to the corresponding consumer within the target time, and transferring the delivery for the corresponding consumer to a neighboring deliverer when the specific deliverer cannot deliver the corresponding dietary food to the corresponding consumer.

Moreover, the delivery target establishment unit sets a wake-up time of the consumer as a start time of the target period in a case in which the dietary food is for a breakfast meal.

Furthermore, the delivery target establishment unit sets the end time of the target period as a meal time of the corresponding consumer, sets time before the maximum time interval determined in advance from the end time as a start time of the target period, and sets a later time among the wake-up time and the start time of the maximum time interval, if there are all of the wake-up time and the start time of the maximum time interval, as the start time of the target period.

Additionally, the delivery schedule unit determines a priority of a consumer to be delivered next from a current location with respect to consumers of delivery destinations of each deliverer, calculates delivery completion time at the current location with respect to the deliverer of each consumer, provides the highest priority when a first principle that the delivery completion time of the corresponding consumer should be time before the end of the target period of each consumer and a second principle that the delivery completion time of the corresponding consumer should be time after the start of the target period of each consumer are satisfied, and provides the next priority when the first principle is satisfied but the second principle is not satisfied.

In addition, the delivery schedule unit calculates the delivery completion time DCT (p,k) for a consumer k of a deliverer P by the following equation:

[DCT(p,k)=T₀+MT(p,k)+DTT(p,k), [Equation 1]

wherein T₀ is current time, and MT(p,k) and DTT(p,k) respectively indicate moving time and delivery task time of the deliverer p to the delivery destination of the consumer k.

Moreover, in a case in which there are two or more consumers who satisfy all of the first and second principles or there are two or more consumers who satisfy the first principle but do not satisfy the second principle, the delivery schedule unit obtains a priority by using an interval among the moving time, the corresponding delivery completion time, and the end time of the target period of the corresponding consumer, and determines detailed priorities according to the obtained priority.

Furthermore, the delivery schedule unit calculates a priority P(p,k) for a consumer k of a deliverer P by the following equation:

$$[ P ( p , k ) = {w}_{1} \frac{MT ( p , k )}{{\sum }_{j = 1}^{N} MT ( p , j )} + {w}_{2} \frac{T 3 ( p , k ) - {T}_{0}}{{\sum }_{j = 1}^{N} ( T 3 ( p , j ) - {T}_{0} )} , {w}_{1} + {w}_{2} = 1 ] ,$$

wherein T₀ is current time, W₁ and W₂ represent a weighted value determined in advance, N represents the number of consumers that the deliverer p does not yet deliver in the delivery zone, and T3 (p,k) represents the end time of the target period.

Additionally, the food transfer processing unit detects a consumer having possibility that the corresponding deliverer cannot deliver within the target period (hereinafter, called ‘transfer target consumer’), obtains the delivery completion time of the corresponding consumer when a consumer having the highest priority at the current time is selected and delivery for the corresponding consumer is completed, excludes the consumer completed in delivery, finds a consumer for the next delivery after obtaining priorities for the remaining consumers based on the obtained delivery completion time, simulates a process that the corresponding deliverer delivers the dietary foods to corresponding consumers in order by repeating the process of finding the next consumer, and performs the simulation till finding the predetermined maximum number of consumers to detect the transfer target consumer.

Moreover, the food transfer processing unit virtually registers the transfer target consumer as a consumer to whom a neighboring deliverer will deliver dietary food, obtains priorities of consumers of the neighboring deliverer, and transfers the transfer target consumer to the neighboring deliverer in a case in which the transfer target consumer is contained in a list of priority determined in advance.

Furthermore, the food transfer processing unit determines not only whether or not the transfer target consumer is contained in a list of priority determined in advance but also whether or not the neighboring deliverer can deliver the dietary food to the corresponding transfer target consumer within the target period of the transfer target consumer and has types of the corresponding transfer target food in stock, simulates the entire process of delivering the food to the transfer target consumer, and checks whether or not the neighboring deliverer can deliver the dietary food to the transfer target consumer within the target period of the transfer target consumer and whether or not the neighboring deliverer has the dietary food for the transfer target consumer in stock.

As described above, the food delivery system tailored to a consumer's eating patterns according to the present invention delivers food by determining priorities of delivery according to a consumer's eating patterns, so that a consumer can receive dietary food at the nearest time to the meal time when the consumer wants to eat before the meal time, thereby maintaining freshness of the dietary food in the best condition.

### BRIEF DESCRIPTION OF THE DRAWINGS

The above and other objects, features and advantages of the present invention will be apparent from the following detailed description of the preferred embodiments of the invention in conjunction with the accompanying drawings, in which:

FIG. 1 is a block diagram of an overall configuration of a food delivery system tailored to a consumer's eating patterns according to the present invention;

FIG. 2 is a block diagram illustrating a configuration of the food delivery system tailored to a consumer's eating patterns according to an embodiment of the present invention;

FIG. 3 is an exemplary view illustrating constituent foods of the dietary food according to an embodiment of the present invention;

FIG. 4 is an exemplary view illustrating the classification of the dietary food by types and capacity of the food according to an embodiment of the present invention;

FIG. 5 is an exemplary view illustrating dietary food by days of the week according to an embodiment of the present invention;

FIG. 6 is a view illustrating a screen for setting an eating pattern according to an embodiment of the present invention;

FIG. 7 is a view illustrating a screen displaying a map in a delivery zone and a delivery list according to an embodiment of the present invention;

FIG. 8 is a view illustrating a screen displaying a delivery destination (or a consumer) in the delivery zone according to an embodiment of the present invention;

FIG. 9 is a view illustrating a delivery list of the delivery destination (or the consumer) according to an embodiment of the present invention;

FIG. 10 is a view illustrating a screen displaying a delivery destination (consumer) for a transfer according to an embodiment of the present invention;

FIG. 11 is a view illustrating a screen displaying a list of neighboring deliverers according to an embodiment of the present invention; and

FIG. 12 is a view illustrating a screen displaying a virtual delivery list of the neighboring deliverers according to an embodiment of the present invention.

### DETAILED DESCRIPTION OF THE PREFERRED EMBODIMENT

Hereinafter, embodiments of the present invention will be described in detail with reference to the accompanying drawings.

In the following description of the present invention, the same parts are denoted by the same reference numerals, and a repeated description thereof will be omitted.

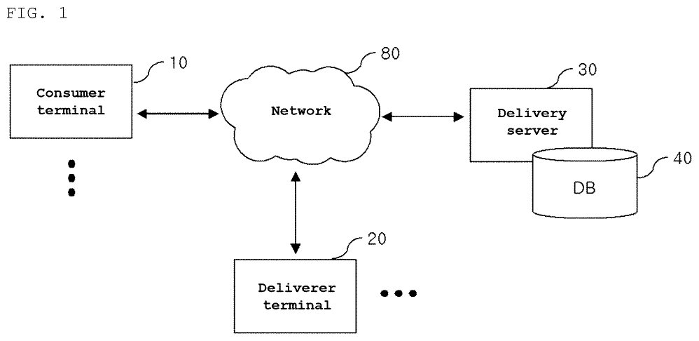

First, referring to FIG. 1, an overall configuration of a food delivery system tailored to a consumer's eating patterns according to the present invention will be described.

As illustrated in FIG. 1, the food delivery system tailored to a consumer's eating patterns according to the present invention includes a consumer terminal 10 used by a consumer, a deliverer terminal 20 used by a deliverer, and a delivery server for providing a dietary food delivery service. Additionally, the food delivery system tailored to a consumer's eating patterns according to the present invention further includes a database 40 for storing consumer information, delivery information, and the like. In addition, the consumer terminal 10, the deliverer terminal 20, and the delivery server 30 are connected with one another via a network 80 to perform data communication.

First, the consumer terminal 10 is a computing terminal used by a consumer, and is a terminal having computing functions, such as a smart phone, a personal computer (PC), a notebook, a phablet, a tablet PC, and the like. In particular, the consumer terminal 10 is a terminal in which web browsers, applications, mobile applications, or the like are installed and executed.

In particular, a client (not shown) interworking with the delivery server 30 is installed in the consumer terminal 10 to receive a food delivery service by linking the corresponding client with the server 30. In addition, preferably, the corresponding client and the delivery server 30 may be built into a client-server system in order to distribute the functions of the entire system according to the performance of the client or traffic capacity with the server. Accordingly, in the following description, the consumer terminal 10 performs work by a consumer (in conjunction with a server) through a corresponding client (or application).

In detail, the consumer terminal 10 accesses the delivery server 30 to order dietary food delivery. Dietary food is delivered regularly. As an example, a consumer can get regular deliveries of various types, such as once a day, twice a day, three times (Monday, Wednesday, and Friday) a week, and the like.

In addition, the consumer terminal 10 may select the capacity and type of the dietary food by the amount and calories of the food. Preferably, the dietary food may be packaged by one meal, and each dietary food is classified for each capacity. Furthermore, the dietary food may be classified into a plurality of types according to the type of the food.

Moreover, the consumer terminal 10 sets a desired delivery arrival time or the like, inputs life information related to the consumer's wake-up time, meal time, and the like, and transmits the life information to the delivery server 30. In this case, the life information can be input by the consumer personally, or assumed by the consumer terminal 10 automatically. For example, the consumer terminal 10 can transmit the wake-up time of an alarm application installed in the consumer terminal as the consumer's wake-up time. Alternatively, the consumer terminal 10 can collect meal information of the consumer (by input of the consumer's meal result) through a diet management application installed in the consumer terminal 10, and transmit the collected meal information to the delivery server 30.

Next, the deliverer terminal 20 is a mobile terminal having computing functions, such as a smart phone, a personal computer (PC), a notebook, a phablet, a tablet PC, and the like. In particular, the deliverer terminal 20 is a terminal capable of being executed by a mobile application which is installed therein.

Especially, a client (not shown) interworking with the delivery server 30 is installed in the deliverer terminal 20, and the corresponding client and the server 30 interwork with each other to use a food delivery service or a delivery guidance service. In addition, preferably, the corresponding client and the delivery server 30 are built into a client-server system, and distribute the functions of the entire system according to the performance of the client, traffic capacity with the server, and the like. Accordingly, in the following description, operations performed by the deliverer terminal 20 is performed through the corresponding client (or the application) by a deliverer (in conjunction with the server).

Specifically, the deliverer terminal 20 is registered as a deliverer and is allocated a delivery zone (or a delivery sector).

In addition, the deliverer terminal 20 senses a location thereof, and transmits the sensed location information to the delivery server 30 in real time. Accordingly, the delivery server 30 can find the locations of all deliverers.

Moreover, the deliverer terminal 20 receives delivery information, such as a consumer who will receive food, an address of the consumer, delivery time, and the like, from the delivery server 30. Especially, the deliverer terminal 20 receives delivery schedule information, such as a delivery path or priorities of delivery, for foods to be delivered (hereinafter, called ‘delivery food’), which are allocated to the deliverer. Accordingly, the deliverer can efficiently deliver the foods allocated thereto according to the delivery schedule information received from the delivery server 30.

Furthermore, the deliverer terminal 20 may receive delivery food (or transfer food) from a deliverer of a neighboring delivery destination. In this instance, preferably, the deliverer terminal 20 may deliver the delivery food allocated to the deliverer instead of the transfer food to the corresponding delivery destination. That is, the deliverer terminal 20 receives the transfer delivery information from the delivery server 30. The deliverer delivers the transfer food according to the transfer delivery information.

In addition, the deliverer terminal 20 receives meeting information, such as a meeting place, meeting time, and the like, to meet the deliverer of the neighboring delivery destination (hereinafter, called ‘neighboring deliverer’). That is, the deliverer moves to the meeting place according to the meeting information to meet the neighboring deliverer, and receives the transfer food from the neighboring deliverer.

Next, the delivery server 30, which is a server for providing a food delivery service, provides the service by interworking with the consumer terminal 10, the deliverer terminal 20 or a client thereof. In particular, the delivery server 30 and the client may be constructed according to a configuration method of a server-client system. That is, functions of the entire system may be distributed according to the performance of the client, traffic capacity with the server, and the like.

In detail, the delivery server 30 receives and registers information on dietary food to be delivered regularly from a manager terminal (not shown). The dietary food is packaged by one meal. Additionally, the dietary food is classified according to the type of food and capacity (or calories).

Meanwhile, each dietary food is classified according to the type and capacity of food, but the number of types of food items according to the type and capacity of food is limited. For example, the dietary food is limited to two food items and three capacities, namely, the number of food items of the dietary food is limited to six.

Moreover, the delivery server 30 receives a membership registration from the consumer terminal 10 or a consumer, and receives an order for delivery of dietary food. The dietary food is ordered to be delivered regularly. Furthermore, the delivery server 30 receives a selection of the food items according to the food type and capacity of the dietary food.

In addition, the delivery server 30 receives meal information, such as meal time of the consumer from the consumer terminal 10. In this instance, the meal information may be directly input by the consumer or may be automatically collected by the consumer terminal 10.

Furthermore, the delivery server 30 generates target delivery information of each consumer according to delivery order information and meal time of the consumer. The target delivery information includes target time to deliver the dietary food to the consumer and food items (or food types) to be delivered at the corresponding time. The target time is a time limit to deliver the dietary food to the corresponding consumer, and the dietary food must be delivered to the corresponding consumer before the target time.

Additionally, the delivery server 30 registers each deliverer, and registers deliverer information and information on the deliverer terminal 20 of the corresponding deliverer. In addition, the delivery server 30 receives location information from the deliverer terminal 20 to find the location of each deliverer terminal 20 or each deliverer.

Moreover, the delivery server 30 classifies delivery zones (or delivery sectors) and allocates the delivery zones to each deliverer. In a case in which the consumer's address (delivery destination) is within a specific delivery zone, the dietary food for the consumer is allocated to the deliverer of the corresponding delivery zone.

In addition, the delivery server 30 generates delivery schedule information of each deliverer and transmits the delivery schedule information to each deliverer terminal 20. In particular, the delivery server 30 generates schedule information by reflecting delivery states and deliverer conditions in real time, and transmits the generated schedule information to each of the deliverer terminals 20 in real time.

In this instance, the delivery server 30 establishes a delivery schedule for each deliverer to deliver dietary food the delivery schedule to each consumer as close as possible to meal time (or target time) of each consumer according to target delivery information for each consumer.

Next, the database 40 includes a food order DB 41 for storing order information, delivery target time, and the like of the dietary food of the consumer, a delivery information DB 42 for storing deliverer information, delivery zones, and dietary food information that the deliverer holds, and a schedule DB 43 for storing information on the delivery schedule. However, the configuration of the database 40 is just a preferred embodiment, and may have another structure according to a database construction theory in consideration of ease and efficiency in access and search.

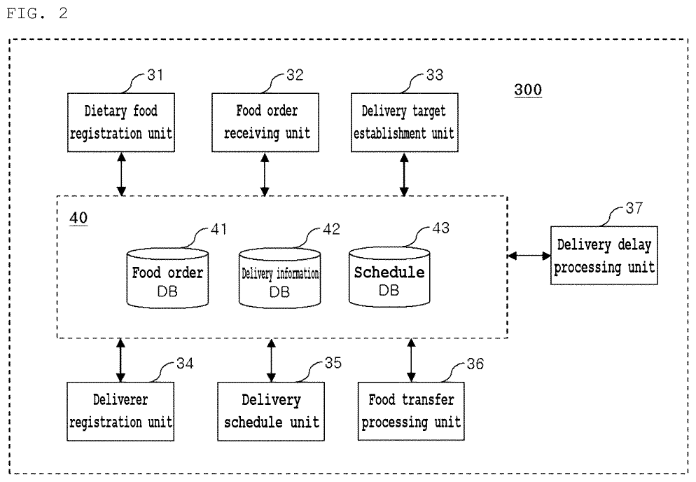

Next, referring to FIG. 2, a configuration of the dietary food delivery system 300 according to an embodiment of the present invention will be described in more detail. The food delivery system according to the present invention may be implemented by a server-client system including a client and a delivery server 30 as described above.

As illustrated in FIG. 2, the dietary food delivery system 300 according to an embodiment of the present invention includes: a dietary food registration unit 31 for registering dietary food; a food order receiving unit 32 for receiving a delivery order of a consumer and collecting eating patterns; a delivery target establishment unit 33 for establishing a delivery target for each consumer; a deliverer registration unit 34 for setting and registering a deliverer and a delivery zone; a delivery schedule unit 35 for generating a schedule for delivery of the consumer's dietary food; and a food transfer processing unit 36 for transferring the dietary food to a neighboring deliverer. Additionally, the dietary food delivery system 300 according to an embodiment of the present invention further includes a delivery delay processing unit 37 for processing a delay in a case in which the delivery is delayed by missing the target time.

First, the dietary food registration unit 31 registers information on the dietary food (or dietary food information). The dietary food is registered or stored in advance by a manager or the like.

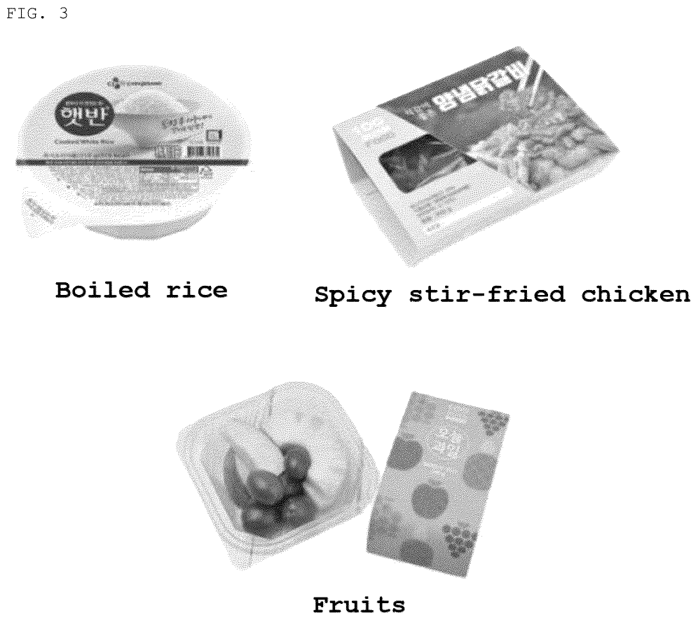

Preferably, the dietary food is packaged by one meal. In addition, as illustrated in FIG. 3, the dietary food for one meal may be composed of a plurality of food items. In FIG. 3, the dietary food for one meal is composed of boiled rice (carbohydrate), spicy stir-fried chicken (protein), fruits (vitamin+sugar), and the like.

Furthermore, the dietary food information includes food name (or product name), food type, capacity (or calorie), main food, and food item information. The food name indicates the title of the corresponding dietary food. The food item information represents information on food items constituting the corresponding dietary food. For example, one dietary food includes food items, such as boiled rice, bean-sprout soup, Jabchae (mixed dish of glass noodles, stir-fried vegetables, and shredded meat), Kimchi, and the like.

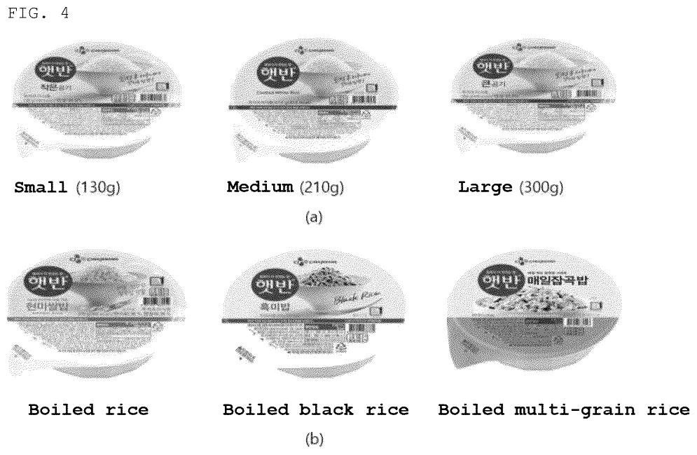

Additionally, as illustrated in FIG. 4, the dietary food is classified according to the type and capacity (or calories) of food. Each dietary food is classified according to the type and capacity of food, but the number of types of food items according to the type and capacity of food is limited. For example, the dietary food is limited to two food items and three capacities, namely, the number of food items of the dietary food is limited to six.

Especially, the dietary food registration unit 31 classifies the capacity of the dietary food or the type by the capacity thereof according to the amount and calories of food. For example, the dietary food is classified into a large size, a medium size, and a small size by capacity, or into 500 kcal, 750 kcal, 1,000 kcal, or the like by calories.

In addition, the dietary food registration unit 31 classifies the dietary food into a plurality of types according to the food type of the dietary food. For example, the dietary food may be classified into a vegetarian diet, a meat diet, and the like, or into beef main diet, chicken main diet, and the like according to main dishes.

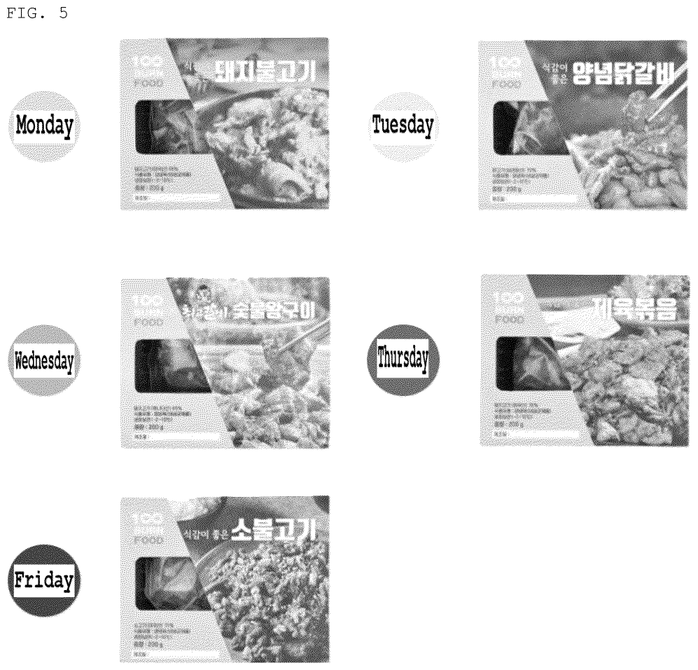

In addition, the dietary food registration unit 31 may vary types of food items of the dietary food for each meal. For instance, two types of food items are provided for breakfast menu and six types of food items are provided for lunch menu on a specific date. Moreover, as illustrated in FIG. 5, the number of types of food items may be varied by days and dates.

Next, the food order receiving unit 32 receives an order of dietary food from the consumer terminal 10 or a consumer. In this instance, the food order receiving unit 32 collects eating patterns of the corresponding consumer.

Furthermore, the food order receiving unit 32 receives delivery order information, such as food types, capacity of foods, a dietary cycle, and the like. Since the dietary food is classified according to food types and capacity, types of food items are determined by food type and capacity. The dietary cycle means a regular meal interval or period for the corresponding dietary food. For example, the dietary cycle is composed of breakfast/lunch/dinner, breakfast/dinner, breakfast, etc. per day, and composed of Monday/Wednesday/Friday, Monday to Friday,

Saturday to Sunday, and the like. That is, the dietary cycle is not set to a specific time, but is set by time zones, such as breakfast/lunch/dinner, days of the week, and the like.

In addition, the food order receiving unit 32 collects eating patterns (life patterns related to meals) of consumers. In this instance, the eating patterns are personally input by the consumer terminal 10 or automatically collected.

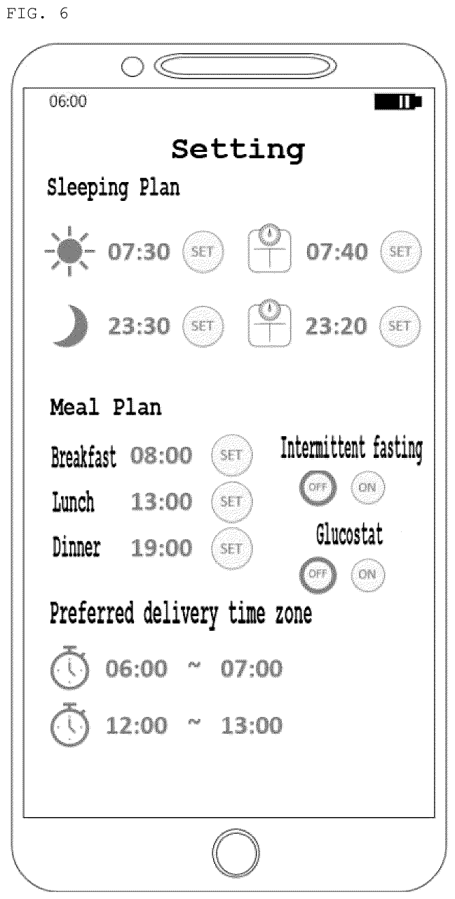

As illustrated in FIG. 6, the food order receiving unit 32 presents a screen for setting the eating patterns of the consumers, and receives each item of the corresponding screen to collect the eating patterns of consumers. The eating pattern is composed of breakfast time, lunch time, and dinner time. Additionally, the eating pattern may include snack time.

The eating pattern may further include wake-up time, bedtime, and the like. The wake-up time and the bedtime are additional life pattern information for controlling noise at the time of delivery. That is, in a case in which the delivery is performed before the wake-up time, noise at the time of delivery may disturb the consumer's sleep. Therefore, if possible, the deliverer does not deliver the dietary food during the bedtime of the consumer to minimize sleep interruption.

In addition, the food order receiving unit 32 automatically estimates the eating pattern of the consumer by the consumer terminal 10. For example, the food order receiving unit 32 can estimate the consumer's wake-up time through the wake-up time set to the alarm application installed in the consumer terminal 10. Alternatively, the food order receiving unit 32 may collect the meal information of the consumer by a diet management application installed in the consumer terminal 10, namely, by inputting meal results of the consumer, to estimate the eating pattern of the consumer by using the collected meal information of the consumer.

Furthermore, the food order receiving unit 32 can estimate the wake-up time by an acceleration sensor of the consumer terminal 10. That is, the food order receiving unit 32 can estimate the wake-up time by operation time of the stopped consumer terminal 10, time when the consumer terminal 10 is change in height, and the like. Alternatively, the food order receiving unit 32 can estimate the wake-up time by the time set for the last alarm among continuous alarms (2˜5 with 1˜5 minute interval), from 5 a.m. to 9 a.m., set to a clock application of the consumer terminal 10. For instance, if the alarm setting time is “5:30 a.m.”, “5:32 a.m.”, and “5:34 a.m.”, or “6:10 a.m.”, “6:15 a.m.”, and “6:20 a.m.”, the food order receiving unit 32 estimates “5:34 a.m.” or “6:20 a.m.” as the wake-up time.

Additionally, the food order receiving unit 32 can estimate the wake-up time through a wearable device connected with the consumer terminal 10, such as a smart band. For instance, the food order receiving unit 32 can estimate the wake-up time by a change in heart rate of the wearable device or by a change in height of the wearable device [height is changed from 20 cm or less to 50 cm or more, and is maintained for 30 seconds or more], and the like.

In addition, the food order receiving unit 32 can estimate the wake-up time through the time when the consumer's weight is measured by a weighting device connected with the consumer terminal 10. That is, the consumer can measure the weight immediately after waking up and immediately before going to bed while performing a weight control through a dietary management. In this instance, the food order receiving unit 32 monitors the weight measurement time of the consumer to extract a weight measurement time pattern. Furthermore, the food order receiving unit 32 estimates a predetermined time interval (for instance, three minutes) before the weight measurement time as the wake-up time. For example, in a case in which the time interval is set for three minutes, the food order receiving unit 32 estimates three minutes before the weight measurement time as the wake-up time. That is, the food order receiving unit 32 estimates it takes three minutes for the consumer to measure the consumer's weight after waking up.

Next, the delivery target establishment unit 33 establishes a delivery target for each consumer by using the dietary food order information and eating patterns of each consumer. The delivery target includes food items to be delivered and target time of the corresponding food items by date or by delivery time zone, namely, by meal.

The dietary food to be delivered is set according to types of the dietary food. The type of the dietary food is determined by type and capacity of food items. That is, the dietary food is not specified by the consumer but is specified by types of food. In a case in which two dietary foods are in the same food type, it doesn't matter which one is delivered. In a general food delivery, a delivery destination (or consumer) is specified according to each product. Therefore, the corresponding product is not delivered to a different delivery destination (or consumer).

Additionally, the target time means a deadline or period to deliver the corresponding dietary food. The deadline is a time limit to deliver the corresponding dietary food, and the period is a section to deliver the corresponding dietary food within the corresponding target time. Preferably, the time limit of the target time may be set as the meal time of the corresponding dietary food.

In FIG. 6, the consumer wakes up at 7:30 a.m., and has a breakfast at 8 a.m. Accordingly, the target time or the target period may be set from 7:30 a.m. to 8:00 a.m. That is, the corresponding dietary food needs to be delivered within the target period (or time zone). Additionally, in a case in which the consumer has lunch at 1:00 p.m., the target time to deliver the dietary food for lunch may be set for 1:00 p.m., so that the dietary food for lunch is delivered before 1:00 p.m.

In addition, preferably, the delivery target establishment unit 33 sets the maximum time interval in advance, and sets a target period so that the dietary food must start to be delivered between a start time of the maximum time interval and the target time. That is, in order to maintain freshness of the dietary food, the maximum time interval is set before the target time. In detail, the target period is set, the end time is the target time, and the start time is time before the maximum time interval from the target time. For instance, in a case in which the maximum time interval is set for two hours, since the target time is 1:00 p.m., the dietary food must start to be delivered after 11:00 a.m. As described above, when the target time and the maximum time interval are set, the target period is set automatically.

Furthermore, preferably, the delivery target establishment unit 33 sets a shorter time or a later time as the start time of the target period if there are the wake-up time and the start time of the maximum time interval in a case of food delivery for breakfast. For instance, in a case in which the consumer wakes up at 5:00 a.m. and has breakfast at 8:00 a.m., the wake-up time is three hours before the breakfast time. Therefore, In this case, 6:00 a.m. which is time before the maximum time interval is set for the start time of the target period.

Additionally, the delivery target establishment unit 33 can set the start time of the target period as the wake-up time in a case of food delivery for breakfast. In FIG. 6, since the consumer wakes up at 7:30 a.m., the start time of the target period for breakfast food delivery is set for 7:30 a.m.

In addition, the consumer can directly input to set time to the delivery target establishment unit 33. As illustrated in FIG. 6, the consumer can input a “preferred delivery time zone”. In this instance, the consumer's preferred delivery time zone may be set as the target period or the target time band of the corresponding consumer.

Meanwhile, the target period is composed of a start time and an end time, and it is more important to keep the end time. That is, in a case in which the dietary food is delivered at 10 a.m., the dietary food is deteriorated in freshness since the consumer has lunch three hours after the delivery. However, in a case in which the dietary food is delivered at 2 p.m., since the dietary food is delivered after the lunch time, the consumer cannot have lunch in fact, and in the worst case, the delivered dietary food may be thrown out.

Next, the deliverer registration unit 34 divides a delivery district into delivery zones or delivery sectors and assigns a deliverer to each delivery zone.

That is, a delivery district is divided into a plurality of delivery zones, and one deliverer takes charge of each delivery zone (or delivery sector). Each deliverer is assigned his or her own delivery zone, and delivers dietary foods in the corresponding delivery zone.

However, in a case in which a deliverer A transfers his or her dietary food to another deliverer B, the deliverer B may deliver the corresponding dietary food to the delivery destination of the corresponding dietary food, that is, to the delivery destination of the deliverer A.

Since the delivery zones are divided from one delivery zone, the two delivery zones may be adjacent to each other. In this instance, one delivery zone adjacent to another delivery zone will be called a neighboring delivery zone, and a deliverer of the neighboring delivery zone will be referred to as a neighboring deliverer.

In addition, the deliverer registration unit 34 registers the deliverer terminal 20 of each deliverer. The information of the deliverer terminal 20 is used to receive location information in real time.

Next, the delivery schedule unit 35 obtains the location information of each deliverer, and generates a delivery path of each deliverer according to the delivery target of each deliverer to schedule the delivery.

In detail, the delivery schedule unit 35 determines priorities of delivery destinations (or consumers) to be delivered next from the current location, with respect to the deliveries of each deliverer. That is, the delivery schedule unit obtains the priorities of the next delivery destinations in this current state and selects and recommends the delivery destination having the highest priority.

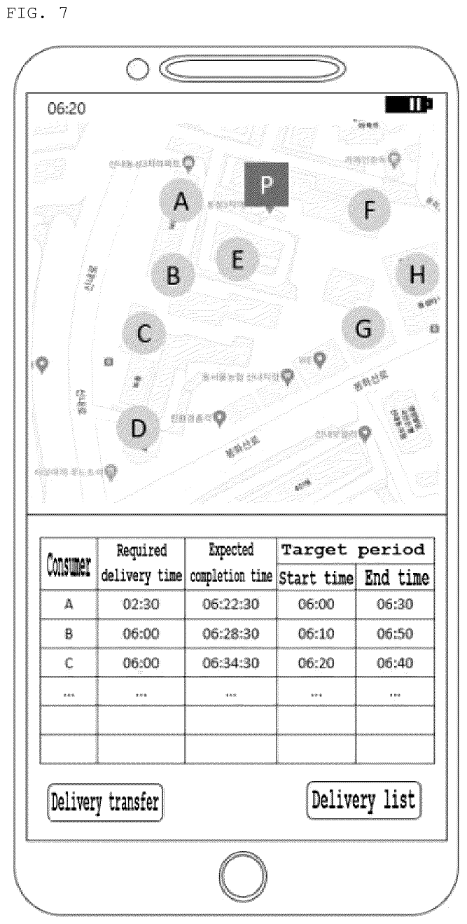

In particular, as illustrated in FIG. 7, the delivery schedule 35 indicates a map in the delivery zone and a delivery destination (consumer) on the map, and at the same time, displays a delivery list. Furthermore, the delivery schedule unit 35 updates and provides the delivery schedule information in real time according to the current state.

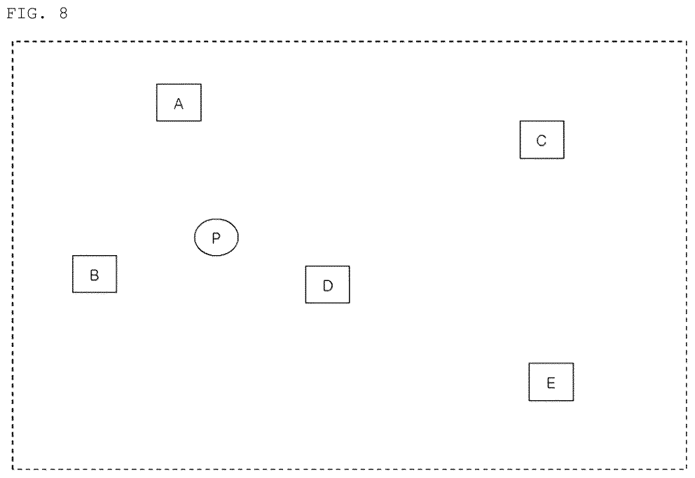

FIG. 8 illustrates the deliveries to deliver dietary foods to delivery destinations (delivery destinations of incomplete delivery) within the delivery zones of a deliverer P. The incomplete delivery destinations are five of delivery destinations A, B, C, D, and E. The delivery destinations A, B, C, D, and E are prioritized.

First, the delivery schedule unit 35 estimates the delivery completion time at the current location with respect to each delivery destination. That is, the delivery schedule unit 35 obtains an expected delivery completion time.

The expected delivery completion time is obtained by adding moving time to a corresponding delivery destination (consumer) and delivery tsk time to the current time. That is, the expected delivery completion time DCT (p,k) of the deliverer P with respect to a consumer k is obtained by the following equation:

DCT(p,k)=T₀+MT(p,k)+DTT(p,k), [Equation 1]

wherein p and k respectively represent a deliverer and a consumer, To is current time, and MT(p,k) and DTT(p,k) respectively indicate moving time and delivery task time of the deliverer p to the consumer k.

The moving time is time required while the deliverer moves from the current location to the corresponding consumer (delivery destination), and the delivery task time refers to time required to perform the delivery task at the delivery destination. Especially, the time required for the delivery task may vary according to the number of floors of a building, installation of an elevator, the direction of an entrance, how to enter a gate, and the like.

Preferably, the moving time is obtained by dividing a moving distance by moving speed. In addition, the moving speed and the delivery task time are estimated by using the accumulated past data. For example, an average moving speed or average delivery task time during a recent specific period (e.g., six months, a year, etc.) in the corresponding delivery zone is obtained, and then, the average value is used as an estimate. Especially, the average delivery task time is obtained by classifying by buildings, apartments, and the like. For instance, in the case of a specific apartment, an average of the past delivery task time measured at the corresponding apartment complex is taken, and then, an expected delivery task time is estimated.

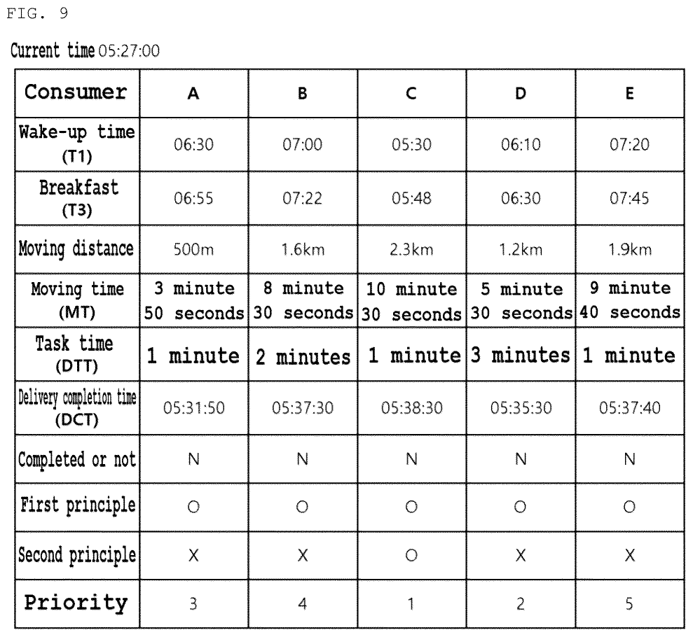

FIG. 9 illustrates estimated moving time, estimated delivery task time, estimated delivery completion time, and the like when the current time is 05:27 and the deliverer P delivers dietary foods to the consumers A, B, C, D, and E. For example, in the case of the consumer A, the moving distance is 500 m. The moving time is estimated by dividing the moving distance by the average moving speed, and the average delivery task time is estimated. In addition, the moving time (MT) of three minutes and fifty seconds and the delivery task time (DTT) of one minute are added to the current time of 05:27:00, so as to obtain the expected delivery completion time (DCT) of 05:31:50.

Next, the delivery schedule unit 35 determines whether the delivery completion time (or expected delivery completion time) of each consumer satisfies the following two principles to determine priorities.

In the first principle, the delivery completion time of the corresponding consumer should be time before the end of the target period of each consumer (or delivery destination). Furthermore, in the second principle, the delivery completion time of the corresponding consumer should be time after the start of the target period of each consumer.

In FIG. 9, with respect to the consumers A to E, since the delivery completion time (or the end time of the target period) of all consumers is before the breakfast time (T3), every all of the consumers A to E satisfy the first principle. However, since only the delivery completion time of the consumer C is the time after the wake-up time or the start time of the target period, only the consumer C satisfies the second principle. Therefore, the consumer C has the highest priority and the corresponding consumer C is selected and recommended as the next delivery destination.

Preferably, the delivery schedule unit 35 preferentially selects and recommends a delivery destination that matches both the first and second principles. Next, in a case in which there is no delivery destination (or consumer) conforming to both the first and second principles, a delivery destination which conforms to the first principle but does not conform to the second principle is preferentially selected and recommended. That is, a delivery destination conforming to both the first and second principles have the highest priority and a delivery destination which conforms to the first principle but does not conform to the second principle has the next priority.

In a case in which there are two or more delivery destinations (or consumers) conforming to both the first and second principles or there are two or more delivery destinations (or consumers) which conforms to the first principle but does not conform to the second principle, priorities are obtained according to the following two criteria, and then, the priorities are determined in detail.

The first criterion is the moving distance (or moving time). The shorter the moving distance (or moving time) is, the higher the priority is. That is, when the moving distance is short, namely, when the moving time is short, the deliverer can deliver dietary foods through the optimal path. Especially, the first criterion takes the priority by the moving time to the corresponding consumer compared with the average moving time of all incomplete delivery destinations in the corresponding delivery zone.

The second criterion is to determine higher priorities when an interval between the delivery completion time and the end time of the target period is short. The shorter a time interval between the delivery completion time and the meal time is, the higher freshness of the dietary food is. Moreover, if the deliverer first delivers food to a consumer coming close to the meal time, it can reduce the possibility of exceeding the meal time (the end time of the target period). Especially, the second criterion takes the priority by the time interval of corresponding consumer compared with the average time interval of all incomplete delivery destinations in the corresponding delivery zone.

Preferably, the first criterion and the second criterion are weighted-averaged to obtain a priority. The lower the priority P (p,k) for the consumer K of the deliverer P is, the higher the order of priority is. The priority is obtained by the following equation:

$$P ( p , k ) = {w}_{1} \frac{MT ( p , k )}{{\sum }_{j = 1}^{N} MT ( p , j )} + {w}_{2} \frac{T 3 ( p , k ) - {T}_{0}}{{\sum }_{j = 1}^{N} ( T 3 ( p , j ) - {T}_{0} )} , {w}_{1} + {w}_{2} = 1 \qquad [ Equation 2 ]$$

wherein W₁ and W₂ represent a weighted value determined in advance, and N represents the number of consumers (delivery destinations) that the deliverer p does not yet deliver in the delivery zone. Additionally, T3 (p,k) represents the end time of the target period (or meal time).

Next, the food transfer processing unit 36 monitors generatability of a consumer that the deliverer cannot satisfy the first principle, and transfers the corresponding consumer or dietary food of the corresponding consumer to a neighboring deliverer (a second deliverer) adjacent to the corresponding deliverer.

In detail, the food transfer processing unit 36 performs simulations as many as the number of foods or consumers previously determined according to the schedule rule, and extracts dietary foods that the deliverer cannot satisfy the first principle to select them as a transfer target as a result of the simulations. That is, in a case in which the dietary food or the consumer having the highest priority at the current time is selected, the food transfer processing unit 36 can obtain the delivery completion time of the corresponding dietary food (or consumer). In that condition, the food transfer processing unit 36 excludes delivery completed dietary foods (or consumers), and finds proprieties of the remaining dietary foods (or consumers) based on the delivery completion time in order to obtain dietary food (or consumer) to be delivered next. As described above, the food transfer processing unit 36 can simulate a process that the deliverer delivers a plurality of delivery destinations (consumers) by repeating the above process of obtaining priorities.

Furthermore, the food transfer processing unit 36 simulates up to the maximum number predetermined (the maximum number of consumers that deliveries were completed), or simulates till finding dietary food which does not conform the first principle. For instance, in a case in which the maximum number of simulations is set to 10, the food transfer processing unit 36 simulates delivery to ten delivery destinations. In this instance, if a delivery destination (consumer) which cannot conform the first principle is generated at the sixth delivery, the food transfer processing unit 36 stops the simulation and selects the corresponding dietary food as a transfer target.

Additionally, the food transfer processing unit 36 processes a delivery delay through the delivery delay processing unit 37 when it is impossible to transfer the dietary food or consumer that the deliverer cannot satisfy the first principle.

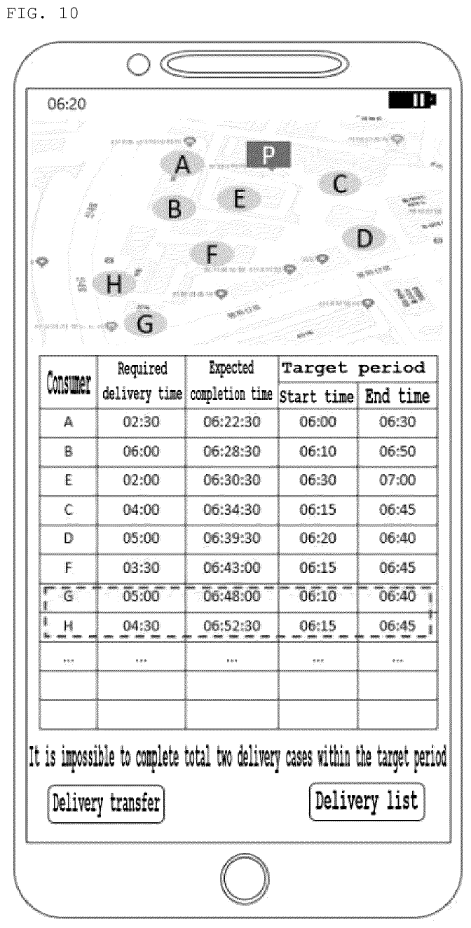

FIG. 10 illustrates a delivery list of delivery destinations that the deliverer cannot satisfy the first principle. For instance, food items G and H are selected as the food items to be transferred.

Meanwhile, the food transfer processing unit 36 makes a schedule for directly transferring dietary food between the deliverer and the neighboring deliverer. In this instance, the food transfer processing unit 36 simulates the delivery process between the deliverer and the neighboring deliverer during a predetermined delivery period of time, for instance, for 30 minutes, and designates a halfway point and halfway time as a meeting place and meeting time when the deliverer and the neighboring deliverer are located at the nearest distance in the corresponding simulation0 zone.

Hereinafter, a process of transferring to a neighboring deliverer will be described.

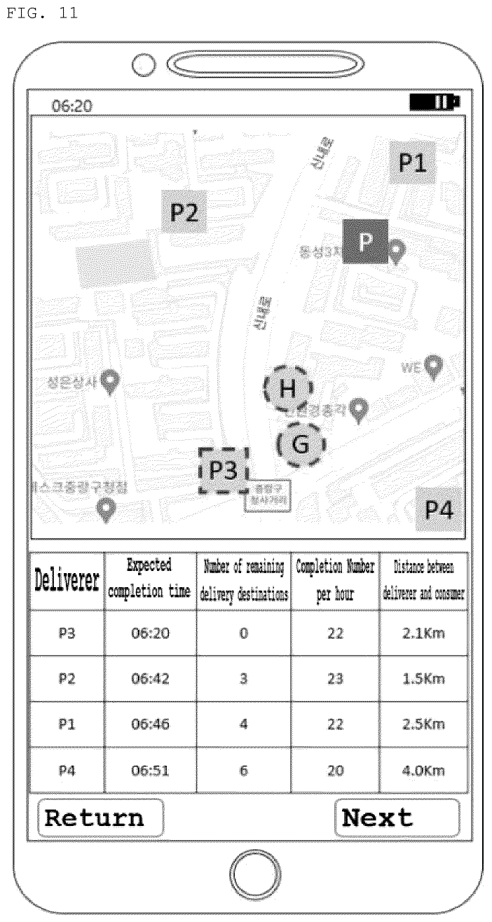

In addition, the food transfer processing unit 36 searches a neighboring deliverer of the deliverer who holds foods to be transferred. In FIG. 11, there are four delivery zones P1, P2, P3 and P4 which are adjacent to the delivery zone of the deliverer P. Additionally, there are two foods G and H to be transferred.

Furthermore, the food transfer processing unit 36 virtually registers the transfer target food as the dietary food of the neighbering deliverer (dietary food to be delivered), and the consumer (transfer target consumer) as the consumer of the neighboring deliverer (or the delivery destination), and obtains priorities of delivery. That is, the food transfer processing unit 36 virtually adds and registers the foods G and H (and the consumer of the foods) to a deliverer P1 as food to be delivered (consumer), which is adding to existing food. Thereafter, the food transfer processing unit 36 obtains priorities with respect to the entire delivery foods (or consumers) of the deliverer P1.

In addition, in a case in which the transfer target food (or transfer target consumer) stands above the predetermined priority order, which means that the transfer target food exceeds minimum order of priority, the food transfer processing unit 36 simulates a process of delivering the transfer target food, and determines whether or not the transfer target food can be delivered within the target time. In this instance, the food transfer processing unit 36 also determines whether or not the neighboring deliverer has food items of the transfer target food in stock.

In FIG. 11, the delivery destinations H and G are closer to the present deliverer P3, and are located closer to the present deliverer P3 than to original deliverer P2. Therefore, the deliverer P3 has a higher priority in the delivery order of the deliverer P3 since the dietary food requires quick delivery and the delivery destination is closer to the deliverer P3. Additionally, a deliverer P2 has a lower priority since being farther away than the deliverer P3, but may have a higher priority if the deliverer P2 has not delivery quantity but the deliverer P3 has lots of urgent delivery quantities.

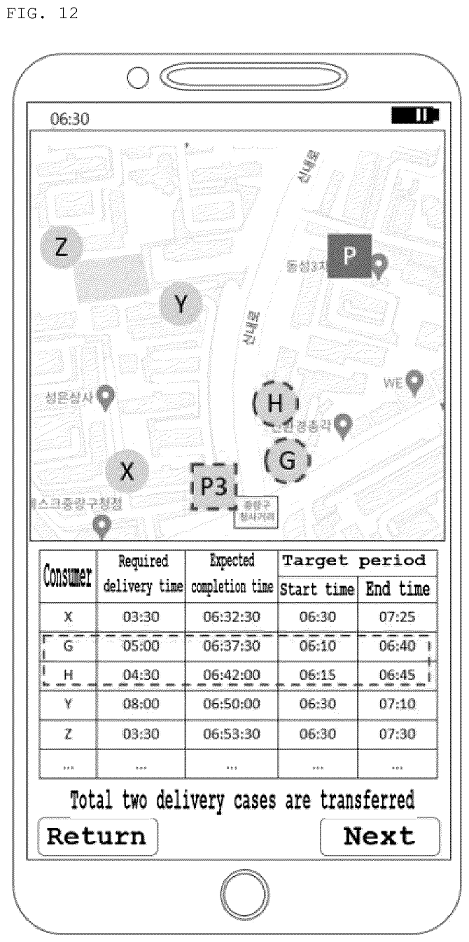

In FIG. 12, the deliverer P3 has foods X, Y and Z to be delivered. In this instance, the foods G and H are additionally assigned to the deliverer P3 virtually, and then, priorities are obtained. In a case in which priorities of the foods G and H are above the minimum order of priority (for instance, in the third rank), the foods G and H are added, and the delivery process is simulated. In this instance, the food transfer processing unit 36 confirms whether or not the foods G or H can be delivered within the target time, and checks whether or not the delivery of the original delivery destination of the deliverer P3 (delivery destination for dietary food or consumer originally assigned to the deliverer P3) breaks the first principle. Thereafter, the food transfer processing unit 36 checks that all food items or food types for the consumers X, Y and Z are in stock. For instance, in a case in which dietary foods are delivered in the order of X, G and H as a result of the simulation, the food transfer processing unit 36 check whether there are food types in stock when the foods G and H are delivered. In a case in which there is food exceeding the target time with respect to the transfer target foods and the original delivery foods of there is no stock, the food transfer processing unit 36 determines transfer delivery is impossible. Meanwhile, in a case in which only one among the foods (delivery destinations) G and H is possible, only the possible food is transferred.

Moreover, preferably, the food transfer processing unit 36 simulates the delivery during a predetermined period of time (or minimum delivery time for simulation). For instance, when the minimum delivery time for simulation is set for 30 minutes, the simulation is performed to proceed delivery for 30 minutes from the current time. This is to secure the time until the deliverer and the neighboring deliverer meet each other and directly transfer the transfer target food.

Furthermore, the food transfer processing unit 36 asks the delivery schedule unit 35 to make a schedule for directly transferring the dietary food between the deliverer and the neighboring deliverer.

Next, in a case in which the target time (end time of the target period) is exceeded, the delivery delay processing unit 37 readjusts the target time or provides compensation according to the penalty.

That is, the delivery delay processing unit 37 presents a time excess list to the deliverer and guides whether to readjust the time by contacting the corresponding consumer.

Furthermore, in a case in which the delivery target time is exceeded, the delivery delay processing unit 37 provides compensation (predetermined compensation) according to the excess. For example, a discount coupon or the like may be provided.

Although the present invention has been described in detail with reference to the exemplary embodiments of the present invention, it is to be understood that the present invention is not limited to the embodiments, but may be changed in various ways without departing from the scope of the present invention.

What is claimed is:

1. A food delivery system tailored to a consumer's eating patterns, the food delivery system comprising:
a dietary food registration unit for registering dietary food classified into a plurality of types;
a food order receiving unit for receiving a delivery order including type of the dietary food and a meal cycle and collecting eating patterns including meal time;
a delivery target establishment unit for establishing target time of the corresponding food including the type of the dietary food to be delivered and a start time and an end time of a delivery period by using the delivery order and the eating patterns of each consumer;
a deliverer registration unit for dividing a delivery district into a plurality of delivery zones and allocating and registering each deliverer to each delivery zone;
a delivery schedule unit for determining priorities of consumers with respect to consumers' delivery destinations assigned to each deliverer and selecting a consumer having the highest priority as a consumer of the next delivery destination; and
a food transfer processing unit for determining whether a specific deliverer can deliver the corresponding dietary food to the corresponding consumer within the target time, and transferring the delivery for the corresponding consumer to a neighboring deliverer when the specific deliverer cannot deliver the corresponding dietary food to the corresponding consumer.

2. The food delivery system according to claim 1, wherein the delivery target establishment unit sets a wake-up time of the consumer as a start time of the target period in a case in which the dietary food is for a breakfast meal.

3. The food delivery system according to claim 2, wherein the delivery target establishment unit sets the end time of the target period as a meal time of the corresponding consumer, sets time before the maximum time interval determined in advance from the end time as a start time of the target period, and sets a later time among the wake-up time and the start time of the maximum time interval, if there are all of the wake-up time and the start time of the maximum time interval, as the start time of the target period.

4. The food delivery system according to claim 1, wherein the delivery schedule unit determines a priority of a consumer to be delivered next from a current location with respect to consumers of delivery destinations of each deliverer, calculates delivery completion time at the current location with respect to the deliverer of each consumer, provides the highest priority when a first principle that the delivery completion time of the corresponding consumer should be time before the end of the target period of each consumer and a second principle that the delivery completion time of the corresponding consumer should be time after the start of the target period of each consumer are satisfied, and provides the next priority when the first principle is satisfied but the second principle is not satisfied.

5. The food delivery system according to claim 4, wherein the delivery schedule unit calculates the delivery completion time DCT (p,k) for a consumer k of a deliverer P by the following equation:
DCT(p,k)=T₀+MT(p,k)+DTT(p,k), and [Equation 1]
wherein T₀ is current time, and MT(p,k) and DTT(p,k) respectively indicate moving time and delivery task time of the deliverer p to the delivery destination of the consumer k.

6. The food delivery system according to claim 4, wherein in a case in which there are two or more consumers who satisfy all of the first and second principles or there are two or more consumers who satisfy the first principle but do not satisfy the second principle, the delivery schedule unit obtains a priority by using an interval among the moving time, the corresponding delivery completion time, and the end time of the target period of the corresponding consumer, and determines detailed priorities according to the obtained priority.

7. The food delivery system according to claim 4, wherein the delivery schedule unit calculates a priority P(p,k) for a consumer k of a deliverer P by the following equation:
$$P ( p , k ) = {w}_{1} \frac{MT ( p , k )}{{\sum }_{j = 1}^{N} MT ( p , j )} + {w}_{2} \frac{T 3 ( p , k ) - {T}_{0}}{{\sum }_{j = 1}^{N} ( T 3 ( p , j ) - {T}_{0} )} , {w}_{1} + {w}_{2} = 1 , \qquad [ Equation 2 ]$$
wherein T₀ is current time, W₁ and W₂ represent a weighted value determined in advance, N represents the number of consumers that the deliverer p does not yet deliver in the delivery zone, and T3 (p,k) represents the end time of the target period.

8. The food delivery system according to claim 4, wherein the food transfer processing unit detects a consumer having possibility that the corresponding deliverer cannot deliver within the target period (hereinafter, called ‘transfer target consumer’), obtains the delivery completion time of the corresponding consumer when a consumer having the highest priority at the current time is selected and delivery for the corresponding consumer is completed, excludes the consumer completed in delivery, finds a consumer for the next delivery after obtaining priorities for the remaining consumers based on the obtained delivery completion time, simulates a process that the corresponding deliverer delivers the dietary foods to corresponding consumers in order by repeating the process of finding the next consumer, and performs the simulation till finding the predetermined maximum number of consumers to detect the transfer target consumer.

9. The food delivery system according to claim 8, wherein the food transfer processing unit virtually registers the transfer target consumer as a consumer to whom a neighboring deliverer will deliver dietary food, obtains priorities of consumers of the neighboring deliverer, and transfers the transfer target consumer to the neighboring deliverer in a case in which the transfer target consumer is contained in a list of priority determined in advance.

10. The food delivery system according to claim 9, wherein the food transfer processing unit determines not only whether or not the transfer target consumer is contained in a list of priority determined in advance but also whether or not the neighboring deliverer can deliver the dietary food to the corresponding transfer target consumer within the target period of the transfer target consumer and has types of the corresponding transfer target food in stock, simulates the entire process of delivering the food to the transfer target consumer, and checks whether or not the neighboring deliverer can deliver the dietary food to the transfer target consumer within the target period of the transfer target consumer and whether or not the neighboring deliverer has the dietary food for the transfer target consumer in stock.
